(19) United States
(12) Patent Application Publication
(10) Pub. No.: US 2024/0058155 A1
(43) Pub. Date: Feb. 22, 2024

(54) ORTHOPEDIC TENSION-REDUCING BRACE

(71) Applicant: Aspen Medical Products, LLC, Irvine, CA (US)

(72) Inventors: Joel Perez, Long Beach, CA (US); Harry Duane Romo, Irvine, CA (US)

(21) Appl. No.: 18/448,756

(22) Filed: Aug. 11, 2023

Related U.S. Application Data
(60) Provisional application No. 63/399,182, filed on Aug. 18, 2022.

(51) Int. Cl. A61F 5/37; A61F 5/02; A61F 5/01

(52) U.S. Cl. CPC A61F 5/3715; A61F 5/028; A61F 5/0109; A61F 2005/0197

(57) ABSTRACT

An orthopedic brace features a knee orthosis including a first de-tensioning assembly and a second de-tensioning assembly. The first de-tensioning assembly includes a first elastic strap situated on a first side of the knee orthosis and the second de-tensioning assembly includes a second elastic strap situated on a second side of the knee orthosis. The first and second elastic straps are communicatively coupled to a lumbar back support brace and a lower leg brace, both separate from the knee orthosis.

### CROSS-REFERENCE TO RELATED APPLICATIONS

This application claims the benefit of priority on U.S. Provisional Patent Application No. 63/399,182 filed Aug. 18, 2022, the entire contents of which are incorporated by reference herein.

### FIELD

Embodiments of the disclosure relate to the field of orthoses. More specifically, one embodiment of the disclosure relates to an orthopedic brace that applies tension laterally and medially along a patient's legs for treating sciatica and providing leg lift assistance.

### GENERAL BACKGROUND

The following description includes information that may be useful in understanding the described invention. It is not an admission that any of the information provided herein is prior art or relevant to the presently claimed invention, or that any publication specifically or implicitly referenced is prior art.

Sciatica is nerve pain manifesting from an injury or irritation to the sciatic nerve, namely a large nerve that made up of five nerve roots: two from the lower back region called the lumbar spine and three from the final section of the spine called the sacrum. The five nerve roots come together to form a right and left sciatic nerve. Both the right and left sciatic nerves run through the hip and buttock regions, down the right and left respective leg and terminates towards the ankle. In some cases, sciatica occurs in response to a nerve condition—an irritation, inflammation, pinching or compression of a nerve in your lower back.

Conventionally, the primary test used to diagnose sciatica is the straight leg raise test. Usually, the patient is lying supine. The affected side leg is lifted at the ankle. Leg elevation is facilitated through flexion at the hip; however, the knee is maintained in an extended position. This creates tension on the sciatic nerve. If additional tension is desired, the foot can be placed in a position of dorsiflexion and even further, the leg may be simultaneously internally rotated. The sum of these positions all increase tension on the inflamed nerve, exacerbating an already painful condition for the patient.

As many people have and continue to experience, the neurologic pain caused by sciatica; it can be debilitating. Efforts are needed to create a solution that is designed to alleviate the pain caused by sciatica, and perhaps provide other ancillary benefits as well.

### BRIEF DESCRIPTION OF THE DRAWINGS

Embodiments of the invention are illustrated by way of example and not by way of limitation in the figures of the accompanying drawings, in which like references indicate similar elements and in which:

FIG. 1A is a perspective front view of a first exemplary embodiment of an orthopedic brace operating as a passive soft exoskeleton.

FIG. 1B is a perspective rear view of the orthopedic brace of FIG. 1A.

FIG. 2A is a perspective front view of an exemplary embodiment of a lumbar support back brace of FIGS. 1A-1B featuring a first brace connection assembly.

FIG. 2B is a perspective rear view of an exemplary embodiment of the lumbar support back brace of FIG. 2A featuring a pulley system and pull tabs.

FIG. 3A is perspective front view of an exemplary embodiment of an intermediary brace of FIGS. 1A-1B operating as a knee orthosis.

FIG. 3B is an exemplary embodiment of the intermediary brace of FIG. 3A placed into an opened state.

FIG. 3C is an exemplary embodiment of the intermediary orthopedic brace of FIGS. 3A-3B illustrating fasteners for maintaining the intermediary orthopedic brace into a closed state when worn.

FIG. 3D is an exemplary embodiment of the intermediary brace of FIGS. 3A-3C positioned in a closed state with optional fastening straps in an unsecured state.

FIG. 3E is an exemplary embodiment of the intermediary brace of FIGS. 3A-3D positioned in the closed state with a first optional fastening strap being positioned into a secured state.

FIG. 3F is perspective side view of an exemplary embodiment of the intermediary orthopedic brace of FIGS. 3A-3E illustrating a first de-tensioning assembly deployed as part of the intermediary brace.

FIG. 3G is perspective side view of an exemplary embodiment of a first transition connection area between a first attachment strap and an elastic strap of the first de-tensioning assembly of FIGS. 3A-3F.

FIG. 311 is perspective side view of an exemplary embodiment of a second transition connection area between a second attachment strap and the elastic strap of the first de-tensioning assembly of FIG. 3G.

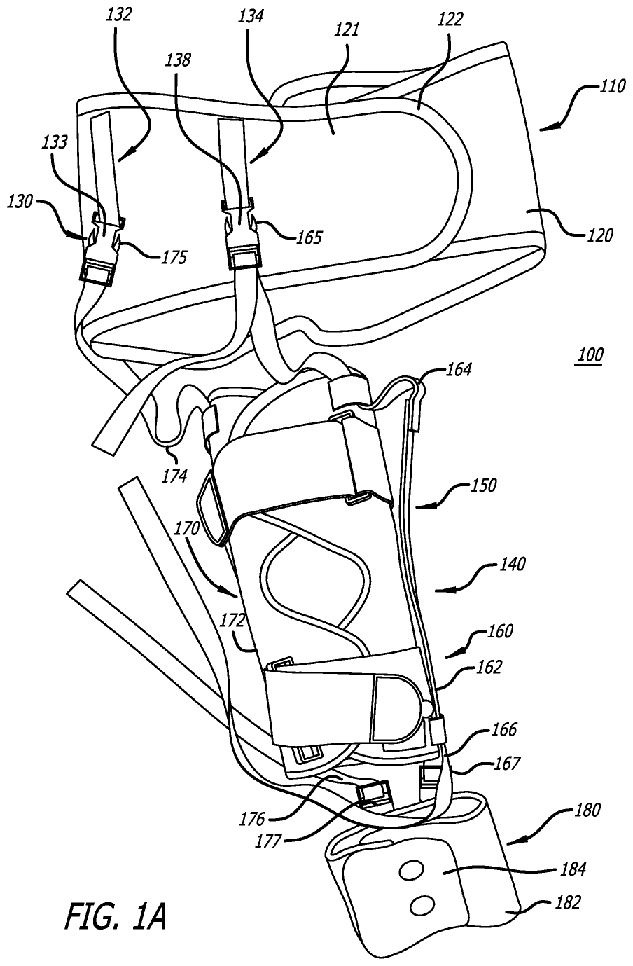

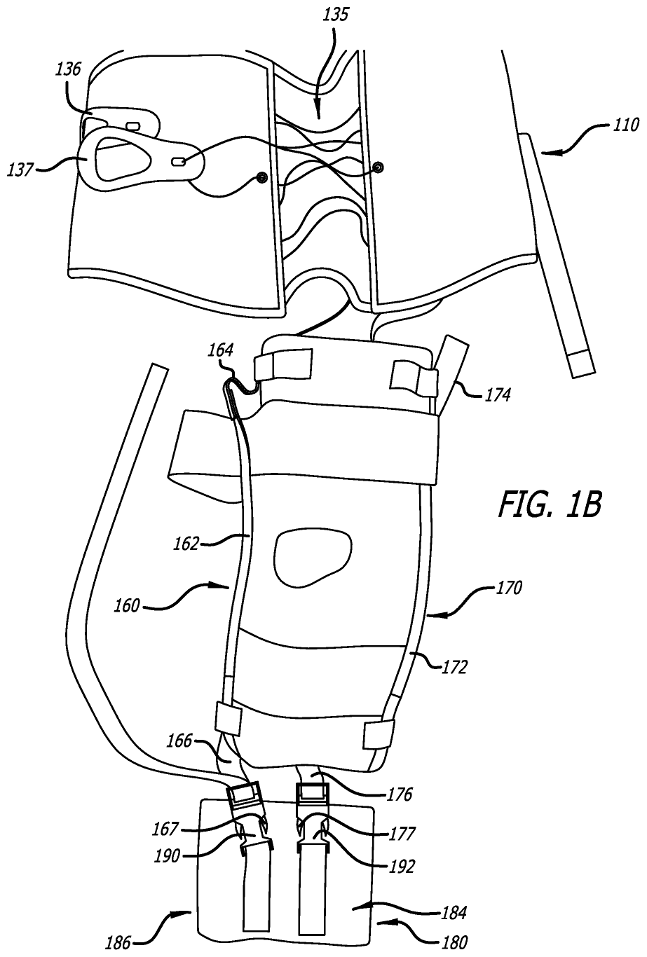

FIG. 4A is a perspective front view of a first embodiment of the lower leg soft exoskeleton of FIG. 1A operating as a calf cuff orthosis.

FIG. 4B is a perspective rear view of an embodiment of the lower leg soft exoskeleton of FIG. 4A operating as a calf cuff orthosis and featuring a second brace connection assembly.

FIG. 4C is a perspective view of the lower leg brace of FIG. 4A positioned in an opened state.

FIG. 5 is a perspective view of the orthopedic brace of FIGS. 1A-4B identifying the brace connection assemblies and their interaction with the intermediary brace operating as a knee orthosis.

FIG. 6A is a perspective side view of a second embodiment of the lower leg brace of FIG. 1A operating as an ankle support orthosis (ASO).

FIG. 6B is a perspective view of the ankle support orthosis (ASO) of FIG. 6A positioned in an opened state.

FIG. 6C is a perspective view of the ankle support orthosis (ASO) of FIG. 6A positioned in a closed state.

FIG. 6D is a perspective rear view of the ankle support orthosis (ASO) of FIG. 6A illustrating the second brace connection assembly.

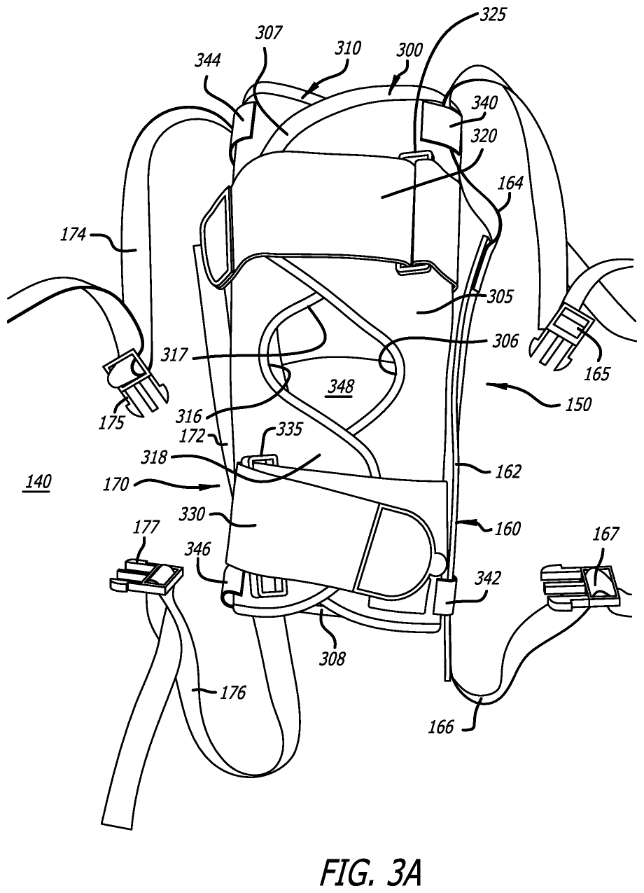

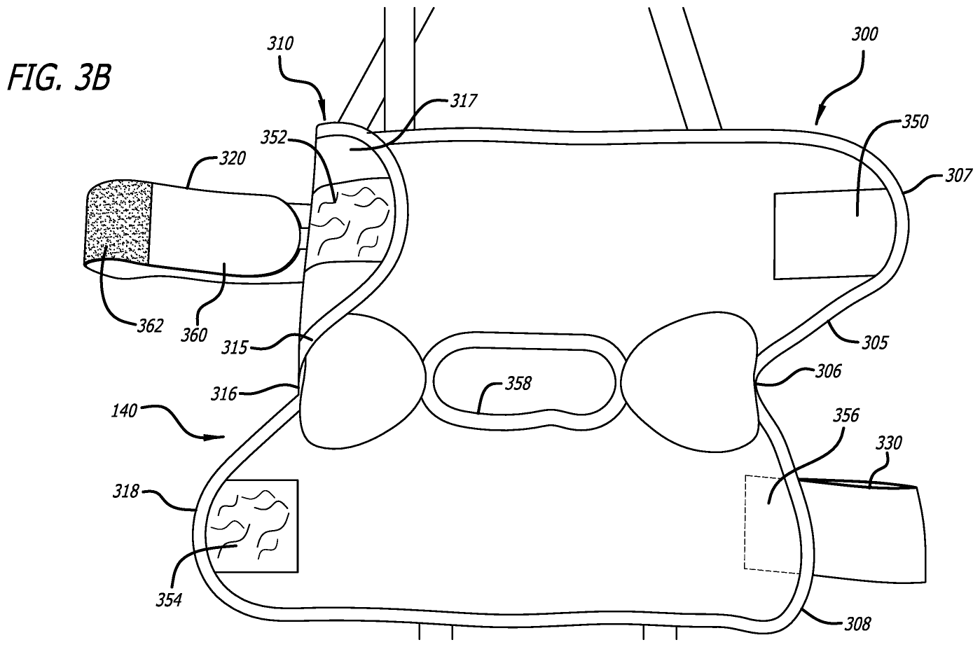

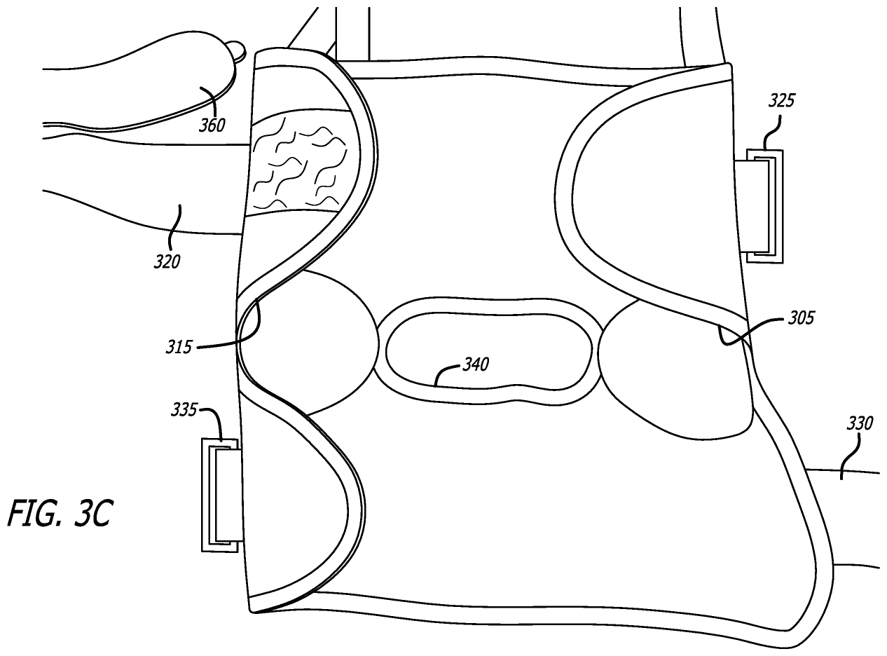

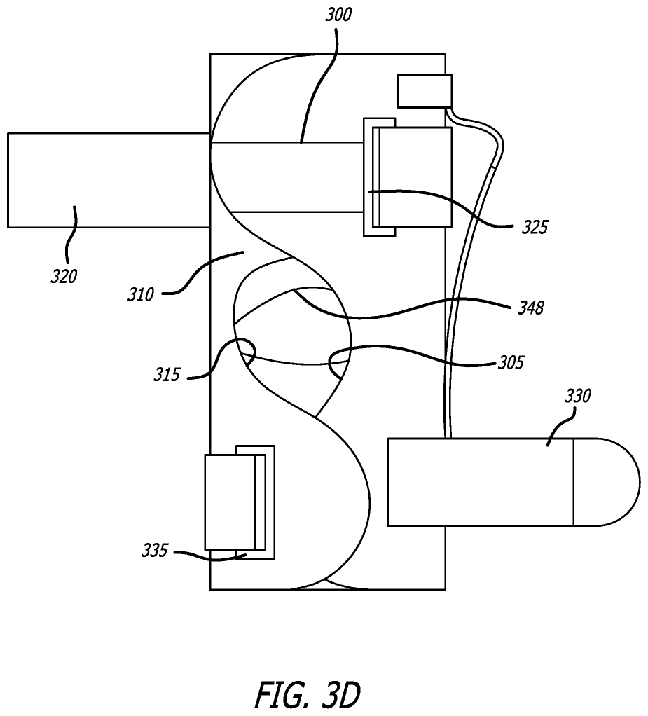

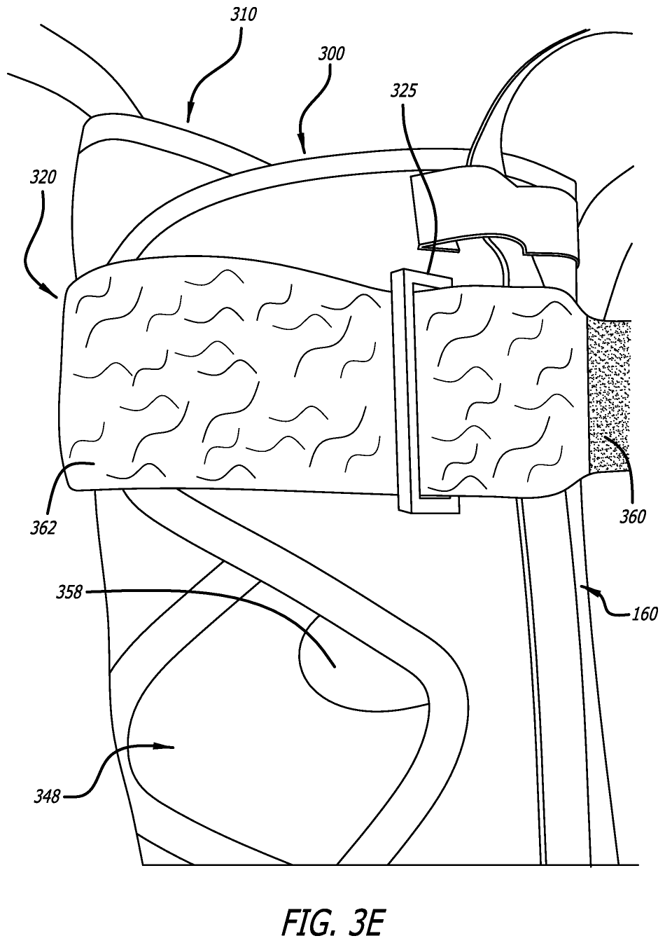

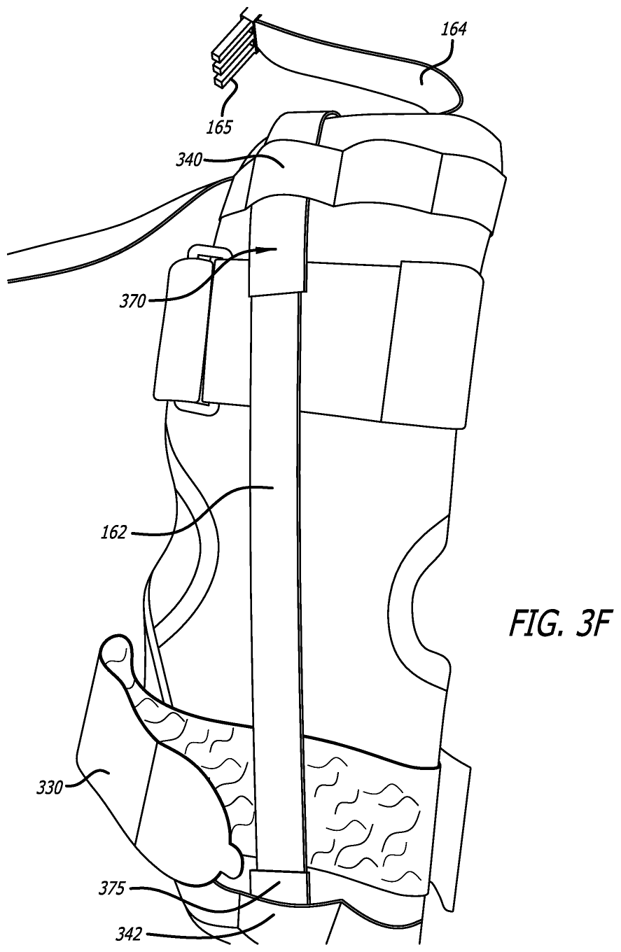

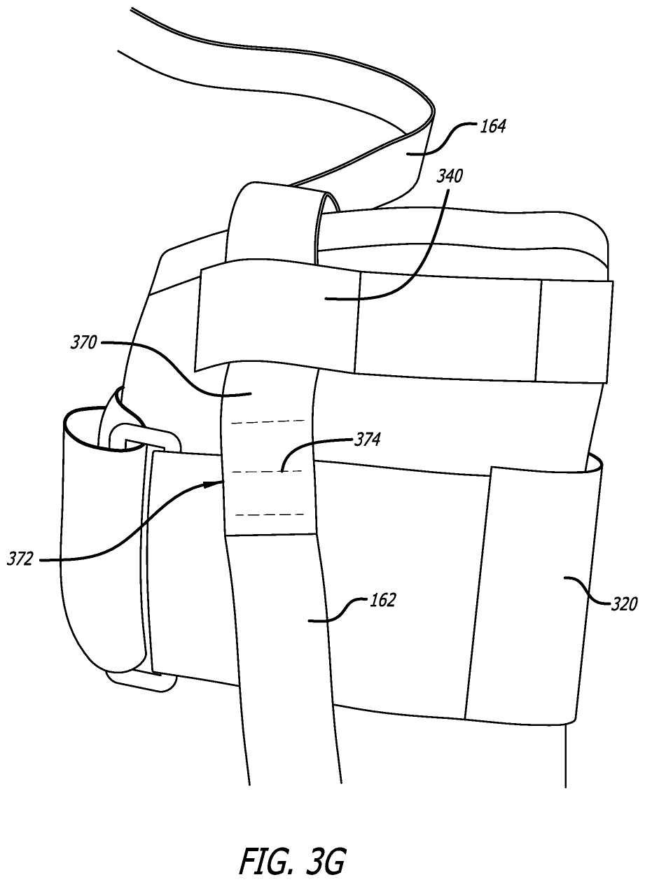

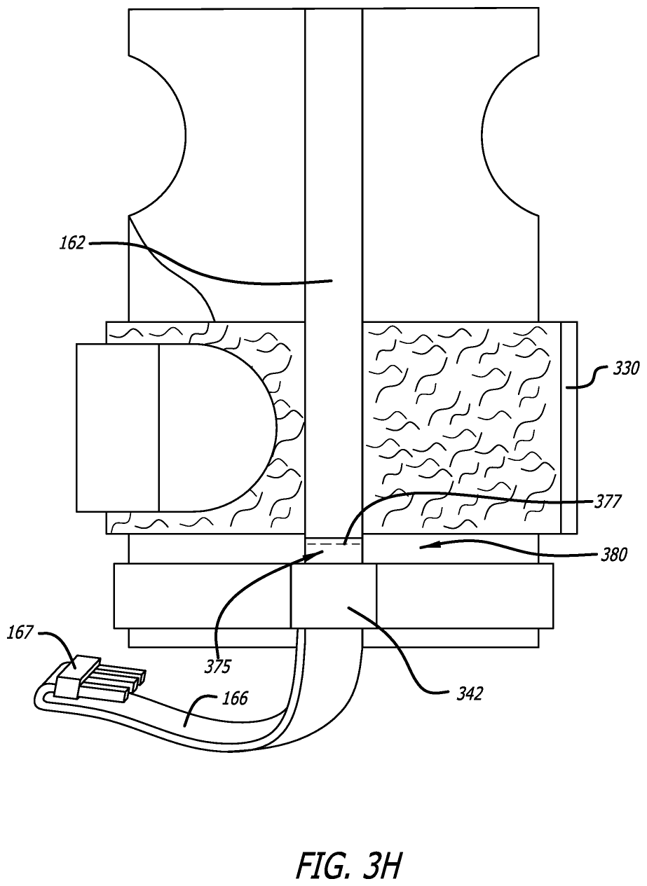

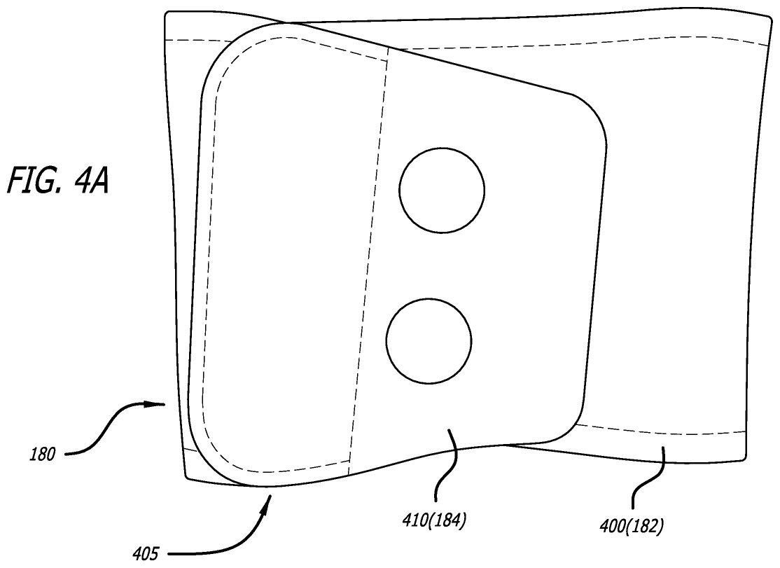

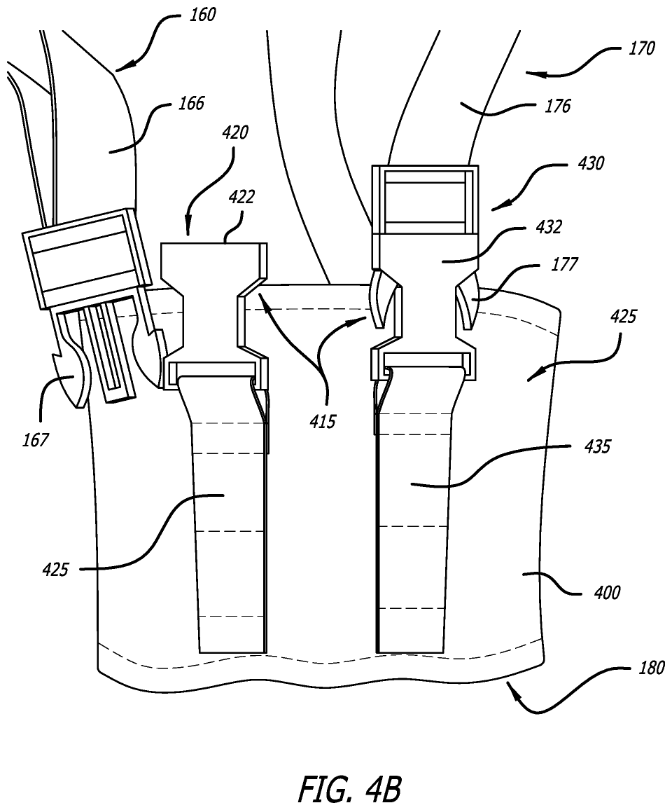

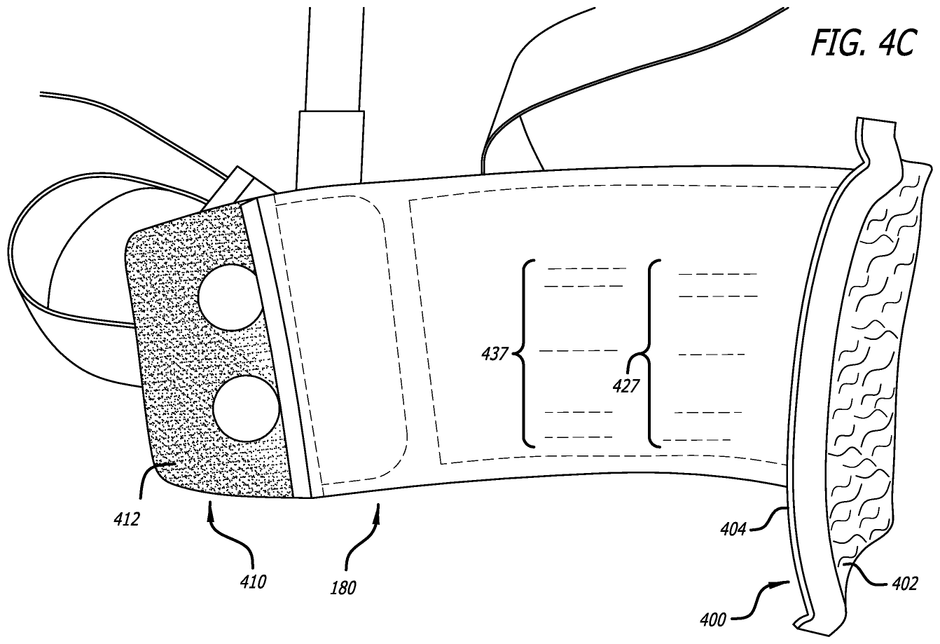

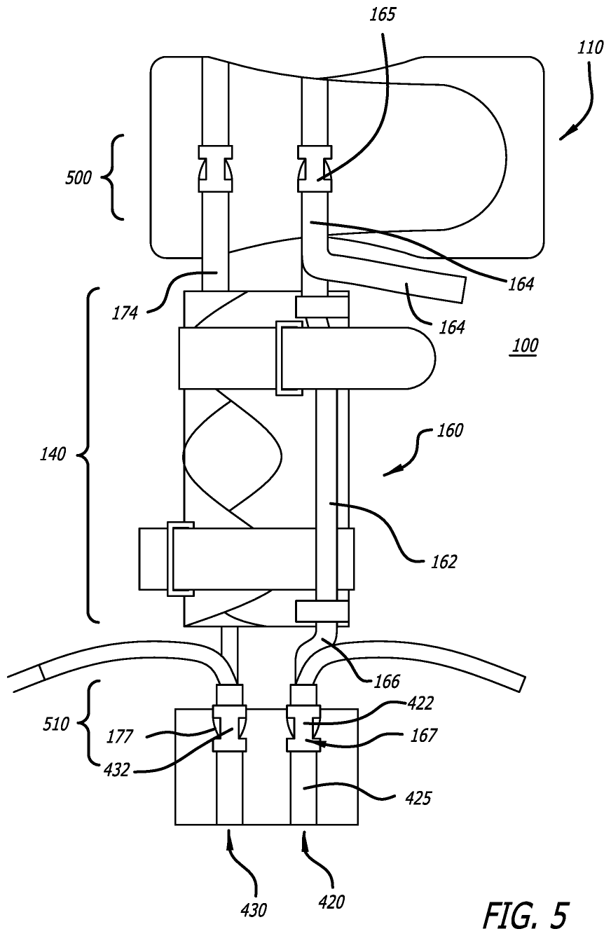

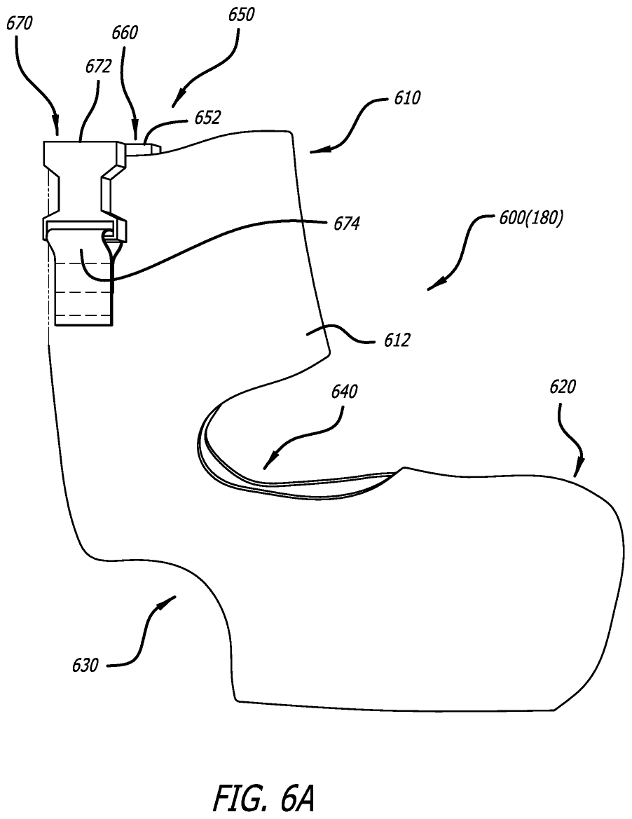

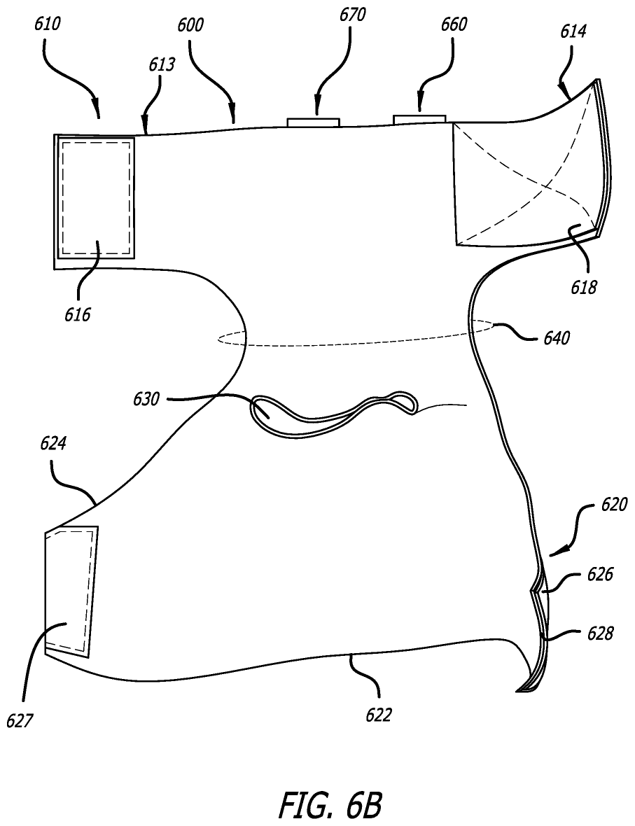

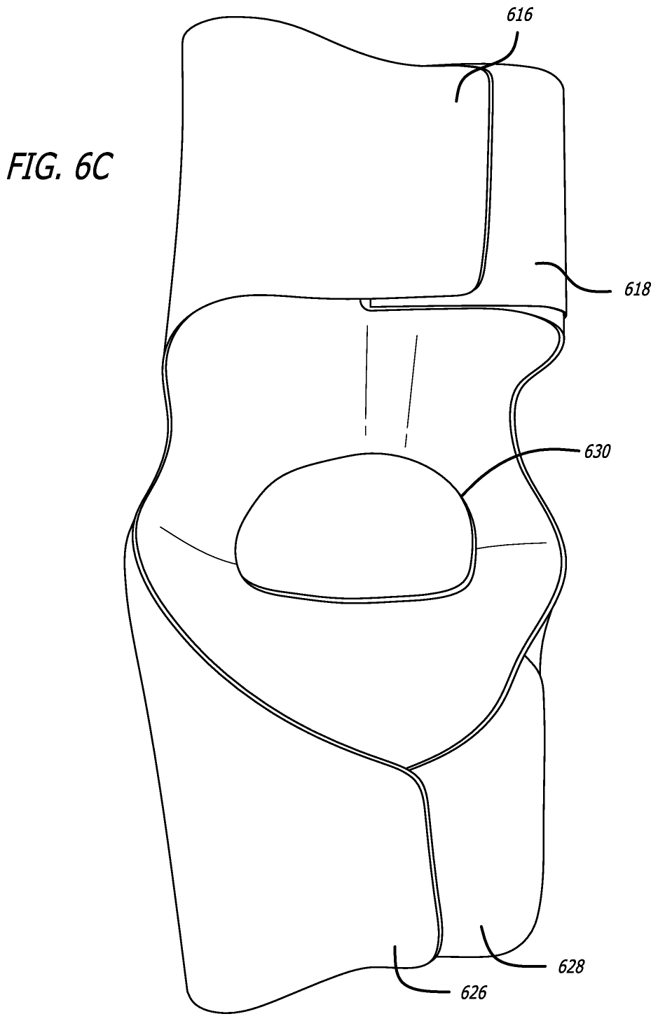

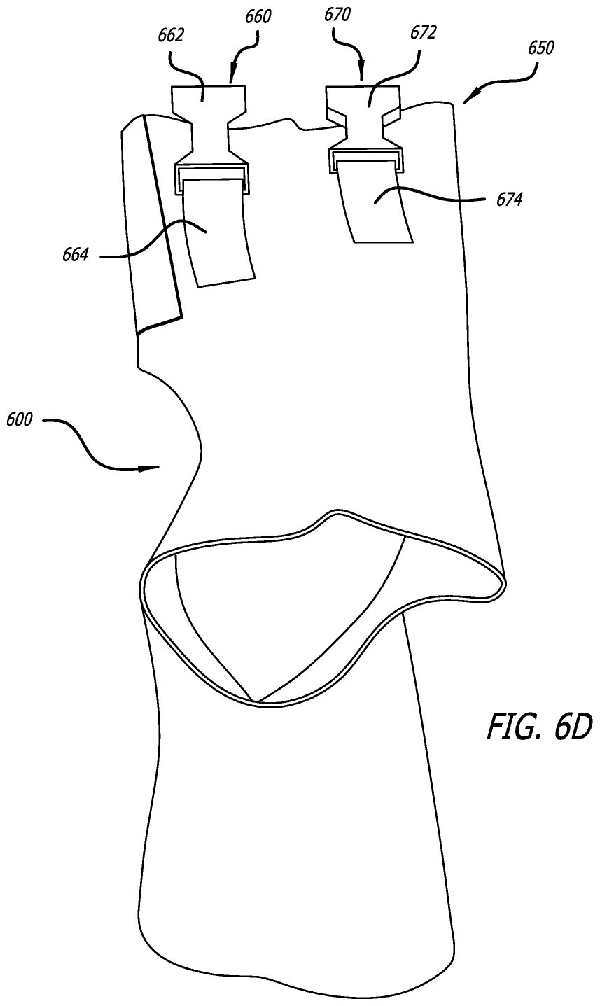

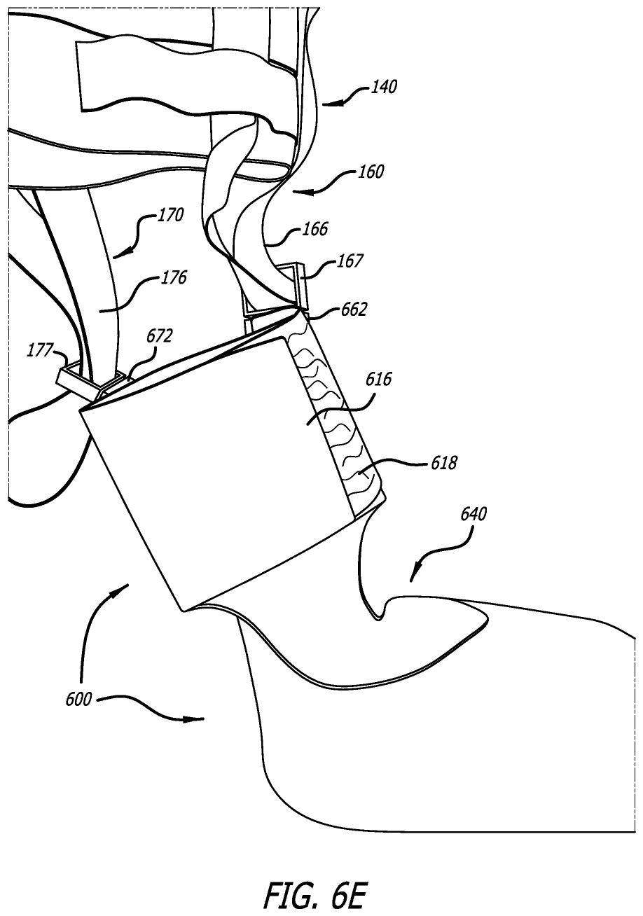

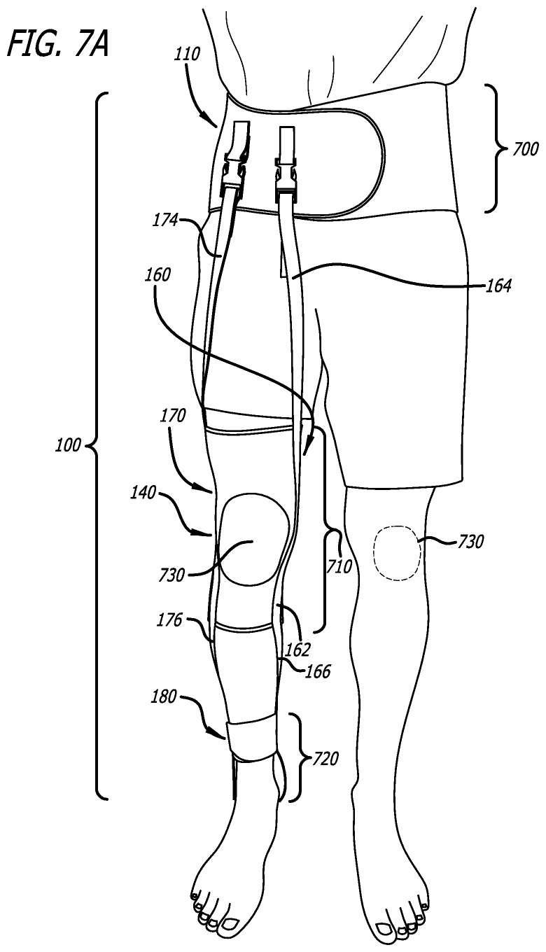

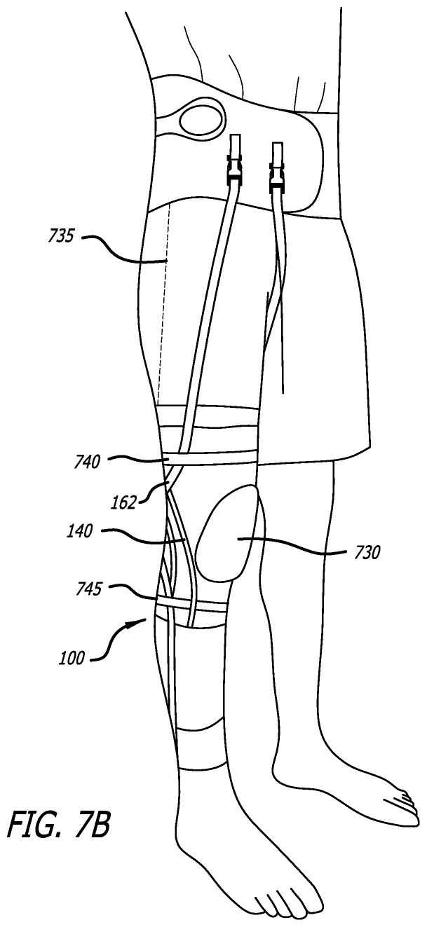

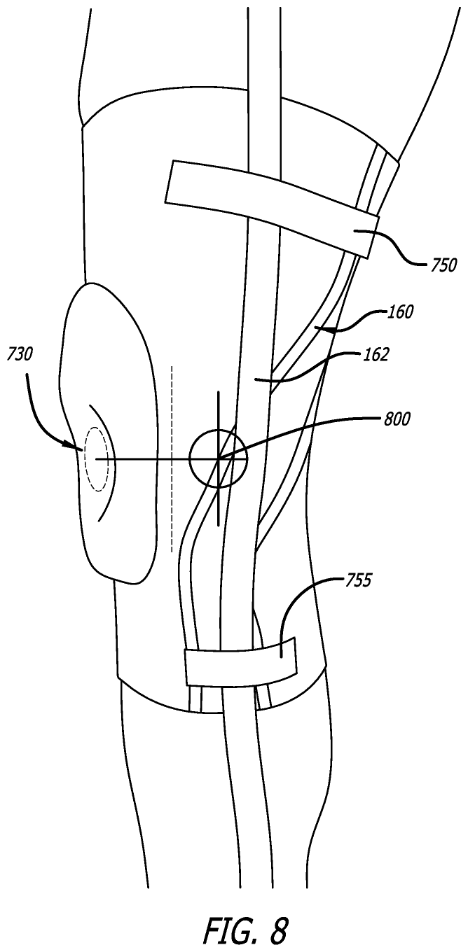

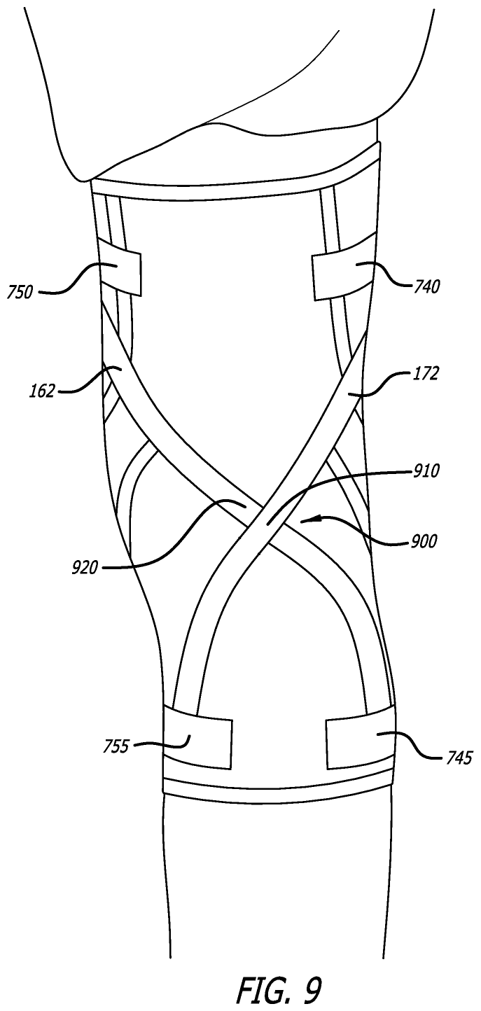

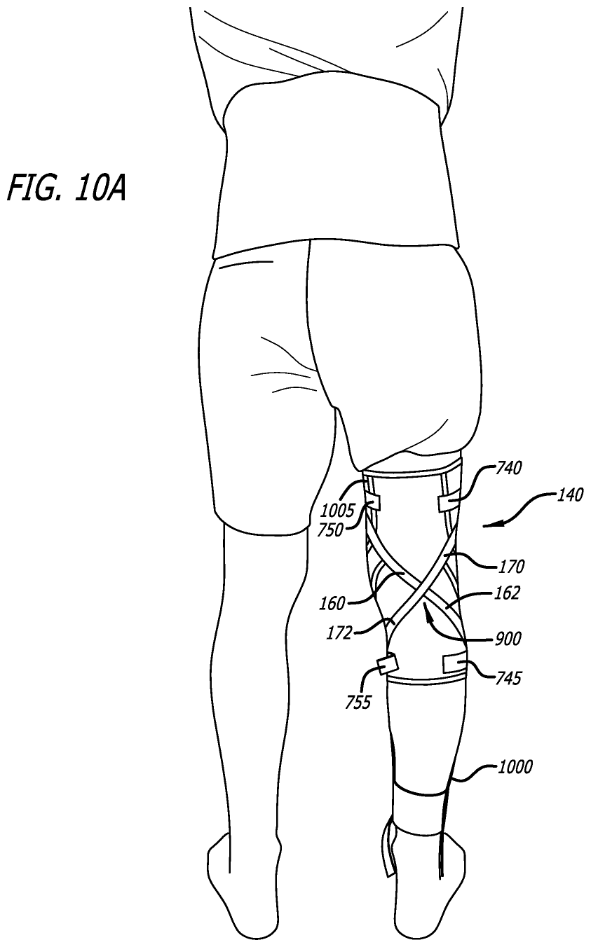

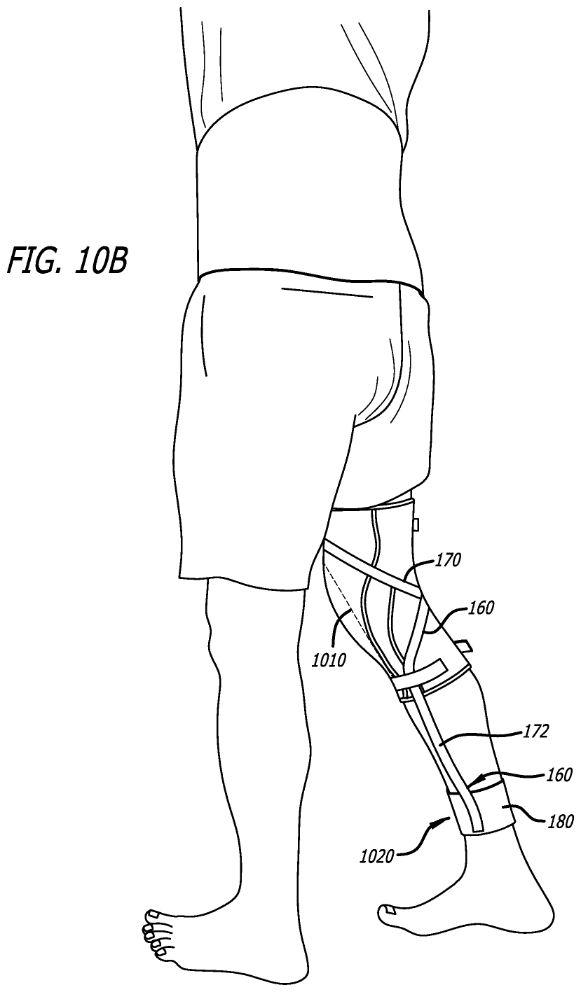

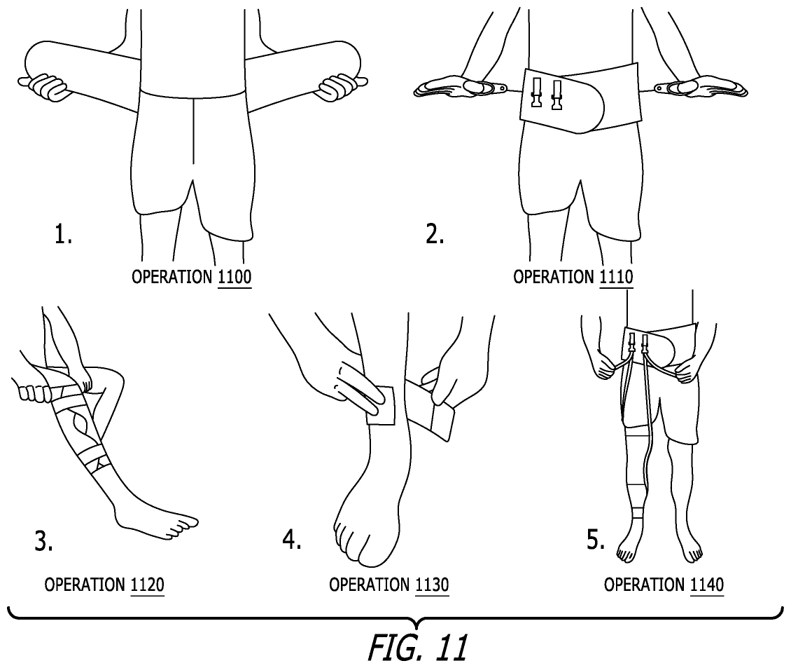

FIG. 6E is a perspective view of a portion of the orthopedic brace illustrating the second brace connection assembly of the ankle support orthosis (ASO) and its interaction with the intermediary brace of FIGS. 3A-31I operating as a knee orthosis.

FIG. 7A is a perspective front view of the orthopedic brace featuring a slide-on knee orthosis with lateral and first de-tensioning assemblies.

FIG. 7B is a perspective view of the orthopedic brace of FIG. 7A.

FIG. 8 is a medial view of a first embodiment of the intermediary brace of FIGS. 7A-7B.

FIG. 9 is a rear view of a second embodiment of the intermediary brace.

FIG. 10A is a perspective rear view of the orthopedic brace of FIG. 9 featuring a rear crossover pattern of the first and second de-tensioning assemblies.

FIG. 10B is a perspective view of the orthopedic brace of FIG. 9 featuring the rear crossover pattern of the first and second de-tensioning assemblies.

FIG. 11 is an exemplary embodiment of the donning process utilized by the orthopedic brace of FIGS. 1A-1B.

### DETAILED DESCRIPTION

Embodiments of the disclosure generally relate to an orthopedic tension-reducing brace, which may operate as soft exoskeleton to create corrective or assistive or resistive forces on the body using textiles, small semi-rigid or rigid structures and expeditious placement of stretch and non-stretch elements across joints to accomplish specific functions, forces, or positioning of body segments. This passive soft exoskeleton solution, referred to as an “orthopedic brace,” features a strap or a collection of straps, at least one strap or a portion of the strap being elastic, positioned behind the knee axis and extending from a lumbar support back brace to a lower leg brace such as a calf cuff or an ankle support orthosis (ASO). The strap or collection of straps (generally referred to as a “tension strap”) is configured to reduce tension on the sciatic nerve thereby reducing the neurologic pain experienced by the patient.

In general, the purpose of the orthopedic brace is to dynamically facilitate a position of the leg which reduces the tension placed on the sciatic nerve. In the case of sciatica, the desired position to de-tension the nerve is that of hip and knee flexion and ankle plantarflexion. This leg position is controlled by routing first and second de-tensioning assemblies featuring elastic straps of sufficient spring constant factor on each side of a leg under tension. The routing for the elastic straps is controlled to pass anterior to the hip joint anatomical axis and posterior to the knee joint anatomical axis. The positioning of the de-tensioning assemblies creates hip and knee flexion moments in sufficient amount to overcome the weight of the leg, thus elevating the knee and foot when relaxed while standing on the unbraced leg allowing the brace-supported side to remain. A patient can still easily overcome this resting position by volitionally extending the hip and knee for instance, during stance. This is possible due to reserve elasticity in the elastic straps and their combined “k” factor does not exceed that which can be generated through contraction of gluteal and quadriceps muscles.

Also, the purpose for two de-tensioning assemblies based on elastic straps is to provide a balanced support on both sides of the leg which, in turn, ultimately affects a resulting transverse plane (rotation of the leg). It is contemplated that the elastic coefficient (k) for an elastic strap of the first de-tensioning assembly may be different than the elastic coefficient associated with the elastic strap of the second de-tensioning assembly. This tension difference could also assist in positioning the internal or external rotational position of the leg in the transverse plane.

Deploying a boot or ASO component, the lower leg brace would be configured to facilitate a de-tensioning position of plantarflexion. This is accomplished by routing the elastic members to connect the boot posterior to the ankle joint. Ideally, the fixation point of the elastic members would be in the region of the Achilles tendon attachment to the calcaneus. This would route the members in the farthest posterior position relative to the ankle joint, maximizing the plantarflexion moment applied.

It is further contemplated that the plantarflexion moment required to alter the position of the foot may be quite low due to the light weight of the foot; not much force is required to facilitate plantarflexion. The amount of force needed would be significantly less than the force required to lift the leg. Thus, a separate tension requirement at the ankle prompting a third, isolated elastic member devoted only to promoting plantarflexion may be implemented.

Lastly, the dynamic de-tensioning assemblies are designed to assist in relieving sciatica pain, as mentioned above, by lifting the leg at rest. The dynamic de-tensioning assemblies may also assist to lift a leg for patients post-stroke or aged. Also, similar dynamic de-tensioning assemblies could be configured to treat similar ailments at other joints of the body such as wrist as one example.

As described below, as an illustrative example, the orthopedic brace includes a lumbar support back brace, an intermediary brace (e.g., knee orthosis) and a lower leg brace are separate and district braces and are coupled together through a tension strap extending through the intermediary brace and terminating at the lower leg brace to the lumbar support back brace. As shown, these braces may be positioned in an opened state and subsequently wrapped around and tightened to a body part of the patient. However, as an alternative embodiment, the intermediary brace and the lower leg brace may be configured as a sleeve to slide over the foot and positioned accordingly.

As this invention is susceptible to embodiments of many different forms, it is intended that the present disclosure is to be considered as an example of the principles of the invention and not intended to restrict the invention to the specific embodiments shown and described.

I. Terminology

In the following description, certain terminology is used to describe aspects of the invention. For example, the terms “assembly,” “component,” and “member” may be construed as a physical structure for use as part of an orthosis. In certain situations, a member may include a rigid component associated with a frame that operates as part of the infrastructure for an orthosis, where the assembly constitutes one or more members. A member may be separate from a soft good (e.g., layers of fabric material connected together) or may not be encased within the soft good.

The term “strap” generally constitutes an interconnect between two termination points. For example, the strap may constitute a piece or pieces of material to be secured at two termination points, where the material may be inelastic (non-stretch) elastic (stretchable), where the degree of elasticity may be selected to achieve a particular therapeutic benefit. Alternatively, the strap may constitute a cable with inelastic and elastic (spring-based) segments.

A “fastener” may be construed as any component that is used to attach different members together or ends of the same component together. An illustrative example of different types of fasteners and fastening techniques may include, but are not limited or restricted to snaps, buttons, clasps, buckles, adhesives, sewing, heat sealing (or melting), gluing, knitting, hook-and-loop, or any other physical coupling techniques.

The term “attach” and other tenses of the term (e.g., attached, attaching, attachment, etc.) may be construed as an act of physically connecting one member to another. The term “coupled” and other tenses of the term (e.g., coupling, couple, etc.) may feature direct attachment as well as indirect attachment via an intermediary between members.

The terms “rigid” or “rigidity” with respect to a member or portion of a member may be construed as the member being configured to resist at least partially bending or deformation. As a result, a rigid member has a structure that may be permanently deformed or broken if bent or twisted (once or repeatedly) by at least 90°. According to this definition, different lengths of a given structure and composition can be rigid at a shorter length, and flexible at a longer length.

Finally, the terms “or” and “and/or” as used herein are to be interpreted as inclusive or meaning any one or any combination. As an example, “A, B or C” or “A, B and/or C” mean “any of the following: A; B; C; A and B; A and C; B and C; A, B and C.” An exception to this definition will occur only when a combination of elements, functions, steps, or acts are in some way inherently mutually exclusive.

II. General Architecture—Orthopedic Brace

Referring to FIG. 1A, a perspective front view of a first embodiment of an orthopedic brace 100 is shown. The orthopedic brace 100 includes a lumbar support back brace 110, an intermediary brace 140 and a lower leg brace 180. As shown, operating as a knee orthosis, the intermediary brace 140 is connected to the lumbar support back brace 110 and the lower leg brace 180 through a sciatica relief assembly 150. The sciatica relief assembly 150 features a first (medial) de-tensioning assembly 160 and a second (lateral) de-tensioning assembly 170.

More specifically, as shown in FIG. 1A, the lumbar support back brace 110 features a belt 120, which is sized to wrap around the waist and lower back of a patient. A first attachment assembly 130 is coupled to an anterior side of a first belt arm 122 of the belt 120. The first attachment assembly 130 features one or more connection points, which may be represented as fastener members, including a first fastener member 132 and a second fastener member 134. As an illustrative embodiment, the first fastener member 132 may features a first (female or male) connector 133 of a first fastener (e.g., quick-release buckle) that is attached to an anterior surface (side) 121 of the first belt arm 122. Similarly, the second fastener member 134 may feature a second (female or male) connector 138 of a second fastener (e.g., quick-release buckle) that is attached to the anterior surface 121. For this embodiment, the lumbar support back brace 110 further includes a pulley system 135 with pull tabs 136 and 137 as shown in FIG. 1B, where each of the pull tabs 136 and 137 is used to tighten a different section of the lumbar support back brace 110, although different types of lumbar braces may be used as a component of the orthopedic brace.

Referring still to FIG. 1A, the intermediary brace 140 includes the sciatica relief assembly 150, which is configured to operate posterior to the knee joint anatomical axis when the intermediary brace 140 is worn. The sciatica relief assembly 150 includes the first de-tensioning assembly 160, which includes an elastic strap 162 that may be positioned along one side (e.g., medial side) of the intermediary brace 140. The elastic strap 162 is coupled to a secondary strap 164, which features a third (male or female) connector 165 of the second fastener for attachment to the second connector 138 associated with the second fastener member 134. Additionally, a fourth (male or female) connector 167 of the second quick-release buckle is attached to a second end of the elastic strap 162. Coupled to opposite ends of the elastic strap 162, the secondary straps 164 and 166 are made of a non-tear material such as nylon, which is inelastic or has an elastic (stretch) composition substantially less than the elastic strap 162. It is noted that these complementary (male and female) connectors for the quick-release buckles may be switched, provided they collectively form fasteners at both the lumbar support back brace 110 and the lower leg brace 180 such as the calf cuff orthosis as shown.

The sciatica relief assembly 150 further includes the second (lateral) de-tensioning assembly 170, which features a construction similar or identical to the first de-tensioning assembly 160 (e.g., situated on the lateral side of the intermediary brace 140). In particular, the second de-tensioning assembly 170 features an elastic strap 172, where a first strap 174 is coupled to a first end of the elastic strap 172 and a second strap 176 is coupled to the other end of the elastic strap 172. The first strap 174 includes a fifth connector 175 complementary to the first connector 133 of the first fastener member 132 while a second strap 176 features a sixth connector 177 for attachment to a complementary buckle at the lower leg brace (e.g., calf cuff orthosis) 180. The lower leg brace 180 operates as a circumference (diameter) adjusting soft good 182 including a loop and hook fastener member 184.

Referring now to FIG. 1B, a perspective rear view of the orthopedic brace 100 of FIG. 1A is shown. In particular, a posterior side 186 of the lower leg brace 180 features fasteners 190 and 192 that are complementary to the connectors 167 and 177 of the first and second de-tensioning assemblies 160 and 170, respectively. The fasteners 190 and 192 are coupled to the posterior side 186 of the lower leg brace 180.

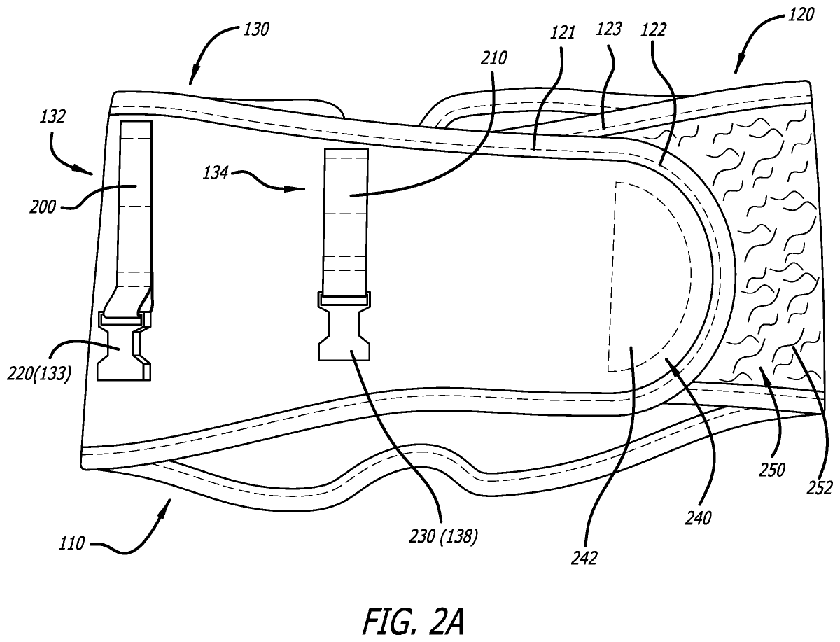

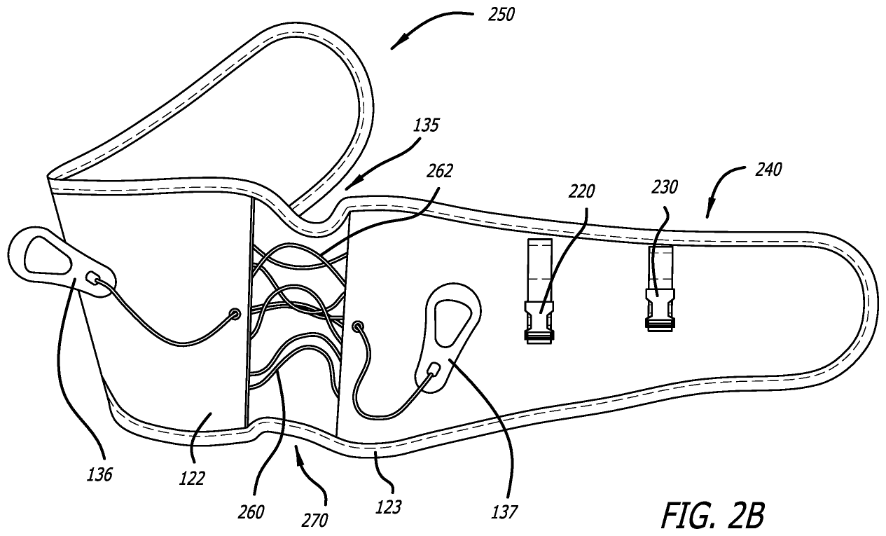

Referring to FIG. 2A, a perspective front view of an exemplary embodiment of the lumbar support back brace 110 of FIGS. 1A-1B is shown. The lumbar support back brace 110 features the first attachment assembly 130, which may include multiple connection points. For example, as shown, the connection points may include a first set (i.e., one or more) of connection points and a second set of connection points. As shown, the first set of connection points may include first fastener member 132, although multiple (two or more) anchor straps may be coupled to the lumbar support back brace 110 to provide an orthotist with an ability to alter placement of the first fastener member 132. Similarly, the second set of connection points may include the second fastener member 134, although multiple (two or more) anchor straps may be coupled to the lumbar support back brace 110 to allow for altered placement of the second fastener member 134.

As shown, according to one embodiment of the disclosure, the first fastener member 132 includes a first anchor strap 200 attached to the anterior side 121 of the first belt arm 122. The first anchor strap 200 includes a first connector 220 (identical to connector 133 of FIG. 1A), which is a portion of the first fastening member 132. Similarly, the second fastener member 134 includes a second anchor strap 210 attached to the anterior side 121 of the first arm 122 of the belt 120. The second anchor strap 210 includes a second connector 230 (identical to connector 138 of FIG. 1A), which is a portion of the second fastener member 134. Herein, the second connector 230 is configured for attachment to the first (medial) de-tensioning assembly 160 while the first connector 220 is configured for attachment to the second (lateral) de-tensioning assembly 170.

The belt 120 includes the first arm 122 and a second arm 123, where the first belt arm 122 is folded to overlay (on top of) the second belt arm 123. Herein, the first belt arm 122 features a hook connector 242 positioned toward an edge 240 of the first belt arm 122 in which the hook connector 242 is configured to attach to a loop material 252 positioned on a top surface 250 of the second belt arm 123.

Referring now to FIG. 2B, a perspective rear view of an exemplary embodiment of the lumbar support back brace 110 of FIG. 2A featuring the pulley system 135 and pull tabs 136-137 is shown. Herein, the pulley system 135 features set of pulleys that are hidden under the first belt arm 122 and the second belt arm 123. Pull tabs 136 and 137 are positioned at the end of cords 270 threaded through the pulleys and terminated proximate to a selected pulley and by tab 136 or 137. Stated differently, a first cord 260 is terminated at the pulley system 135, wound through selected pulleys, and terminated at the pull tab 136. A similar configuration exists for a second cord 262, which is coupled to and terminated at the pulley system 135 and the pull tab 137.

Referring now to FIG. 3A, a perspective front view of an exemplary embodiment of the intermediary brace 140 of FIGS. 1A-1B operating as a knee orthosis is shown. Herein, the intermediary brace 140 may be configured as a soft good constructed as a sheet of material 305 (hereinafter, “base material”) so that, when folded, a first end 300 of the base material 305 partially overlaps a second end 310 of the base material 305. As shown, the first end 300 includes a recessed area 306 positioned opposite a recessed area 316 of the second end 310. A first extension area 307 for the first end 300 of the intermediary brace 140 is positioned to overlay a first extension area 317 of the second end 310. Similarly, a second extension area 318 for the second end 310 of the intermediary brace 140 is positioned to overlay a second extension area 308 of the first end 300. Optional fastening straps 320 and 330 and corresponding strap guide rings 325 and 335 are used for tightening the intermediary brace 140 around the leg of the patient.

As shown, the intermediary brace 140 features the first de-tensioning assembly 160 and the second de-tensioning assembly 170, which are located anterior to the patient's hip joint anatomical axis and posterior to the anatomical knee joint axis. More specifically, the elastic straps 162 and 172 are located behind the knee joint anatomical axis of the patient when the intermediary brace 140 is worn. The first and second de-tensioning assemblies 160 and 170 with elastic straps 162 and 172 is to provide a balanced support on both sides of the leg which, in turn, ultimately affects a resulting transverse plane (rotation of the leg). The tension coefficients (k factors) for the elastic straps 162 and 172 may be the same or different, where a tension difference could also assist in positioning the internal or external rotational position of the leg in the transverse plane.

More specifically, the routing for the elastic straps 162 and 172 may be positioned to pass anterior to the hip joint anatomical axis and posterior to knee joint anatomical axis. The positioning of the de-tensioning assemblies creates hip and knee flexion moments in a sufficient amount to overcome the weight of the leg, thus elevating the knee and foot when relaxed while standing unweighted on the garment-supported side. This is possible due to reserve elasticity in the elastic straps and their combined k factor does not exceed that which can be generated through contraction of gluteal and quadriceps muscles.

To ensure positioning of the elastic strap 162 posterior to the knee joint axis and anterior to the hip joint axis, the first (medial) de-tensioning assembly 160 further features the secondary straps 164 and 166 inserted through guide channel 340 and 342, respectively. Located on an anterior surface of the first end 300 formed by base material 305, the guide channels 340 and 342 prevent movement outside a first parasagittal plane to retain the first de-tensioning assembly 160 between the knee joint and hip joint. As shown, made of nylon or another inelastic material, the secondary straps 164 and 166 are attached to the elastic strap (material) 162 and includes the third and fourth connectors 165 and 167, respectively.

Additionally, to ensure positioning of the elastic strap 172 between the knee joint axis and hip joint axis, the second de-tensioning assembly 170 further features the secondary straps 174 and 176 inserted through guide channel 344 and 346 located on an anterior surface of the second end 310, respectively. The guide channels 344 and 346 restrict movement along a second parasagittal plane to retain the second de-tensioning assembly 170 behind the knee joint axis. As shown, the secondary straps 174 and 176 are attached to the elastic strap 172 similar in manner to the straps 164 and 166 being attached to the elastic strap 162. The secondary straps 174 and 176 include the fifth and sixth connectors 175 and 177, respectively.

Referring now to FIG. 3B, an exemplary embodiment of the intermediary brace 140 (knee orthosis) of FIG. 3A placed into an opened state is shown. In the opened state, the intermediary brace 140 features the first and second ends 300 and 310 of the base material 305 located on opposite sides of the intermediary brace 140. The first end 300 of the intermediary brace 140 features the recessed area 306 positioned between the extension areas 307-308 while the second end 310 features the recessed area 316 positioned between the extension areas 317-318. Herein, an inner surface of the extension area 307 features a fastener 350 (e.g., hook fastener) while an outer (rear-facing) surface of the extension area 317 features a fastener 352 (e.g., loop fastener) complementary to the fastener 350 and coupled together upon wrapping the extension area 317 under the extension area 307. Also, an inner surface of the extension area 318 features a fastener 354 (e.g., hook fastener) while an outer surface of the extension area 308 features a fastener 356 (e.g., loop fastener) complementary to the fastener 354. This enables the extension area 307 of the first end 300 to overlay the extension area 317 of the second end 310 and the extension area 318 of the second end 310 to overlay the extension area 308 of the first end 300.

When the intermediary brace 140 is placed into a closed state as shown in FIG. 3A, an opening 348 for the patient's knee is created for patient comfort and increased mobility. Additionally, an opening 358 is positioned proximate to a center area of the intermediary brace 140 (and aligned with the popliteal fossa) to alleviate restriction of the patient's leg during movement.

As shown in FIGS. 3B-3E, the intermediary brace 140 further includes the first fastening strap 320 configured for positioning over the extension areas 307 and 317 when folded upon each other, threaded through the strap guide ring 325, and looped back for attachment to itself via loop and hook fasteners 360 and 362 placed on the fastening strap 320. A similar architecture is relied upon by extension areas 308 and 318 being folded over each other and fastened together through the fastening strap 330 and strap guide ring 335.

Referring now to FIG. 3F, a perspective side view of an exemplary embodiment of the intermediary brace 140 of FIGS. 3A-3E illustrating the first de-tensioning assembly 160 is shown. A first connection area 370 between the elastic strap 162 and the secondary strap 164 is configured to be aligned with the guide channel 340 while a second connection area 375 between the elastic strap 162 and the secondary strap 166 is configured to be aligned with the guide channel 342. When the connectors 165 and 167 are connected to complementary fasteners within the lumbar support back brace 110 and the lower leg brace 180, during leg movement (e.g., flexion, extension, abduction, or adduction), the elastic strap 162 is positioned and configured to reduce tension placed on the sciatic nerve by creating hip and knee flexion moments through the knee and foot elevation while standing unweighted on the brace-supported side.

Referring to FIGS. 3G-31I, perspective side views of exemplary embodiments of the first (transition) connection area 370 between the elastic strap 162 and the secondary strap 164 and the second (transition) connection area 380 between the elastic strap 162 and the secondary strap 166 forming portions of the first de-tensioning assembly 160 is shown. The first (transition) connection area 370 provides an attachment point 372 between the elastic strap 162 and the secondary strap 164, where the attachment may be accomplished by any fastening mechanism such as a sewn attachment 374 as shown. For this embodiment, the first (transition) connection area 370 is positioned over the optional strap 320.

Similarly, the second (transition) connection area 375 provides an attachment point 377 between the elastic strap 162 and the secondary strap 166, where the attachment may be accomplished by any fastening mechanism. For this embodiment, the second (transition) connection area 375 is positioned over the optional fastening strap 330.

Referring to FIG. 4A, a perspective front view of a first embodiment of the lower leg brace 180 of FIG. 1A is shown. Operating as a calf cuff orthosis, the lower leg brace 180 includes a linear strip of soft goods 400 (like soft goods 182) with a fastener 410 (like fastener member 184) coupled to a first end 405 of the soft good 400. As shown, the fastener 410 may constitute a loop and hook fastener member in which the hook operates as the fastener 410 and the loop fastener portion is part of the soft good 400. Herein, the lower leg brace 180 is positioned around a calf of a patient and tightened using the fastener 410.

Referring now to FIG. 4B, a perspective rear view of the lower leg brace 180 of FIG. 4A operating as a calf cuff orthosis and featuring a second brace connection assembly 415 is shown. The second brace connection assembly 415 includes a third attachment assembly 420 and a fourth attachment assembly 430. According to one embodiment of the disclosure, although not shown, the third attachment assembly 420 and the fourth attachment assembly 430 may be formed through a sewn connection between the secondary straps 166 and 176 and the anchor straps 425 and 435, respectively. As an alternative embodiment, as shown, the third attachment assembly 420 may include a connector 422 and an anchor strap 425 for attachment of the third attachment assembly 420 to the soft good 400. Additionally, the fourth attachment assembly 430 may include a connector 432 and an anchor strap 435 that attaches the fourth attachment assembly 430 to the soft good 400. The connectors 422 and 432 of the first and second attachment assemblies 420 and 430 are adapted for connectivity with connectors 167 and 177 of the secondary straps 166 and 176, respectively. This attachment enables the intermediary brace 140 to be attached to the lower leg brace 180. More specifically this enables the first de-tensioning assembly 160 and a second de-tensioning assembly 170 to be anchored or terminated by the lower leg brace 180.

Referring to FIG. 4C, a perspective view of the lower leg brace 180 of FIG. 4A positioned in an opened state is shown. Herein, the soft good 400 is illustrated with a top surface 402 of the soft good 400 constituting a loop material for connection with the fastener 410, namely a hook material portion 412 of the opening of the fastener 410. The interior surface 404 of the soft good 400 includes stitching patterns 427 and 437 that represent attachments or fastening mechanisms for the anchor straps 425 and 435, respectively.

Referring now to FIG. 5, a perspective view of the orthopedic brace 100 of FIGS. 1A-4B identifying the brace connection assemblies and their interaction with the intermediary brace 140 is shown. Herein, the lower leg brace 180 has been rotated and is illustrating a rear viewpoint of the brace 180 to illustrate the third and fourth attachment assemblies 420 and 430. Herein, the orthopedic brace 100 features two connection regions 500 and 510. The first connection region 500 is represented by quick-release side buckles with a first buckle portion provided by the lumbar support back brace 110 and a second connector being part of the quick-release side buckle be provided by straps 164 and 174 associated with the intermediary brace 140. A second connection region 510 illustrates the connectivity between the connectors 422 and 432 of the lower leg brace (calf cuff) being coupled to connectors 167 and 177 of straps 166 and 176 associated with the intermediary brace 140. Through these connection regions 500 and 510, the first and second de-tensioning assemblies 160 and 170 are communicatively coupled from the waist to the ankle to provide sciatica relief.

Referring now to FIG. 6A, a perspective side view of a second embodiment of the lower leg brace 180 of FIG. 1A operating as an ankle support orthosis (ASO) 600 is shown. The ASO 600 includes an ankle member 610, a foot member 620, and recessed openings 630 and 640 positioned around the heal and the talus of the foot. Herein, the ankle member 610 features another embodiment of a second brace connection assembly 650 that includes the third attachment assembly 660 and the fourth attachment assembly 670. The third attachment assembly 660 includes connector 662 and the fourth attachment assembly 670 includes connector 672, where connectors 662 and 672 are complimentary to the connectors 167 and 177, respectively. As with the first embodiment of the lower leg brace 180, the connectors 662 and 672 are attached to material 612 forming the ankle member 610 via anchor straps 664 and 674. As shown, the third and fourth connectors 662 and 672 are oriented upward for attachment to connectors 167 and 177 to remain so that the first de-tensioning assembly 160 and the second de-tensioning assembly 170 is positioned between the hip joint anatomical axis and the knee joint anatomical axis.

Referring now to FIG. 6B, the ASO 600 of FIG. 6A positioned in an opened state is shown. The recess opening 630 for the heal is illustrated at a center area of the ASO 600 when placed into an opened state. As shown, the ankle member 610 features a first end 613 and a second end 614. The first end 613 includes a fastener 616 to be coupled to a complimentary fastener 618 of the second end 614.

Similar in construction as the ankle member 610, the foot member 620 includes an edge upon which the toes extend through an opening formed by a first end 624 and a second end 626 when attached. The first end of the foot member 620 includes a fastener (hook fastener), which is complimentary with and attached to a fastener 628 located on the second end 626. As shown in FIG. 6C, upon placement of the second end 614 underneath the first end 613 of the ankle brace member 610 and placement of the second end 626 under the first end 624 of the foot brace member 620, the ASO 600 is formed. For this situation, the fastener 616 is attached to the fastener 618 while the fastener 627 is attached to the fastener 628.

Referring now to FIG. 6D, a perspective rear view of the ASO 600 of FIG. 6A illustrating the second brace connection assembly 650 is shown. As provided in greater detail than in FIG. 6A, the third attachment assembly 660 and the fourth attachment assembly 670 are positioned on opposite sides of a sagittal plane of the ASO 600. The connector 662 and the anchor strap 664 provide secure placement of the third attachment assembly 660 in a medial location for communication with the first de-tensioning assembly 160 while the connectors 672 and anchor strap 674 of the fourth attachment assembly 670 is positioned for coupling with the second de-tensioning assembly 170 as shown in FIG. 6E. More specifically, as shown in FIGS. 6D-6E, the connector 662 is configured for coupling to the connector 167 attached to the secondary strap 166 of the intermediary brace 140. The connector 672 is coupled to the connector 177 of the secondary strap 176 that extends from the intermediary brace 140. Based on the positioning of the third and fourth attachment assemblies 660 and 670, the first de-tensioning assembly 160 and the second de-tensioning assembly 170 remain behind the knee joint axis of the patient.

Referring now to FIG. 7A, a perspective front view of the orthopedic brace 100 including the intermediary brace 140, which is illustrated as a knee sleeve being an alternative embodiment to the wrapped knee orthosis shown in FIGS. 3A-31I, is shown. The connection mechanism for the orthopedic brace 100 is identical with the exception to the intermediary brace 140 remaining in a closed state given its sleeve configuration. The orthopedic brace 100 includes the lumbar support back brace 110 operating as a first component 700 of the orthopedic brace 100. Based on connectivity via the secondary straps 164 and 174, the intermediary brace 140 is attached to the lumbar support back brace 110. In particular, the first de-tensioning assembly 160 and the second de-tensioning assembly 170 include elastic straps 162/172 and secondary straps 164/166 and 174/176, which are positioned at a second connection area 710 to provide de-tension of hip and knee flexion and ankle plantarflexion to alleviate tension on the sciatic nerve. The positioning of the elastic strap 162 and 172 (not shown) is behind the knee joint axis 800 (see FIG. 8) which, according to one embodiment of the disclosure, is level with a middle of the patella 730 and posterior to a midline extending laterally from a center of the leg. The orthopedic brace 100 is terminated at a third connection point 720 as represented by the lower leg brace 180 (e.g., calf cuff orthosis).

Referring now to FIG. 7B, a perspective view of the orthopedic brace 100 of FIG. 7A is shown. Herein, the guide channels 740 and 745 are oriented and sewn (attached) on the intermediary brace 140 so that, for this embodiment, the elastic strap 162 remains behind anatomical axis of the knee 730 and anatomical axis of the hip joint 735. It is contemplated that the elastic strap may be positioned lateral along the leg of the patient instead of behind the knee.

Referring now to FIG. 8, a medial view of a first embodiment of the intermediary brace 140 of FIGS. 7A and 7B is shown. Herein, the guide channels 750 and 755 are provided to retain the elastic strap 162 of the first de-tensioning assembly 160 behind the anatomical knee joint axis 800 that is shown level to a middle of the patella 730 and posterior to a central axis of the leg. The guide channels 750 and 755 are not attached to the elastic strap 162, but rather, provides an alignment for the elastic strap 162 so that the strap 162 remains in its position and not moved anterior from the confined openings formed by the guide channels 750 and 755 (i.e., posterior to the knee joint axis 730).

Referring now to FIG. 9, a rear view of a second embodiment of the intermediary brace 140 is shown. Herein, in lieu of the elastic strap 162 and 172 progressing along the sides of the knee area in a linear fashion, the elastic straps 162 and 172 crisscross behind the knee at area 900 so that the elastic strap 162 of the first de-tensioning assembly 160 progresses through the guide channels 750 and 745. Similarly, the elastic strap 172 is configured to propagate through guide channels 740 and 755. As a result, the elastic straps 162 and 172 crisscross so that a portion 910 of one of the elastic straps (e.g., elastic strap 172) overlays a portion 920 of the other elastic strap (e.g., elastic strap 162).

Referring to FIG. 10A, a perspective rear view of the orthopedic brace 100 of FIG. 9 featuring a rear crossover pattern of the first and second de-tensioning assemblies 160 and 170 is shown. Herein, a first end portion of the elastic strap 162 is threaded through guide channels 750 and 755 while elastic strap 172 is passed through guide channels 740 and 752 to create crisscross pattern (i.e., crossover pattern) within the area 900. As a result, one of these elastic straps 162 and 172 will overlay the other, such as elastic strap 172 overlaying elastic strap 162 to create the crisscross pattern. Hence, an upper portion 1005 of the first de-tensioning assembly 160 is at a medial location of the leg while a lower portion 1000 of the first de-tensioning assembly 160 is positioned toward the ankle of the patient. The opposite is true for the second de-tensioning assembly 170. As shown in FIG. 10B, an anatomical axis 1010 of the knee joint identifies the positioning of the joint center in reference to the leg 1020 remains in front of the de-tensioning assemblies 160 and 170. As in FIG. 1A, the lower leg brace (calf cuff) 180 may be used to terminate the de-tensioning assemblies 160 and 170.

Referring now to FIG. 11, an exemplary embodiment of the donning process and utilized by the orthopedic brace 100 of FIGS. 1A-1B is shown. First, the lumbar support back brace is separated and placed in an opened state and attached to the waist of the patient (operations 1100 and 1110). The lumbar support back brace may be tightened as shown. Next, the intermediary brace is positioned over the knee when the intermediary brace is operating as a knee orthosis. As shown, the knee orthosis may be slid over the foot onto the knee or it may be wrapped when the knee orthosis can operate in an opened and closed state (operation 1120). Thereafter, a lower leg brace is attached. This may be accomplished through a calf cuff as shown in which the calf cuff is attached to the lower calf area of the patient. Lastly, the connections may be made while we are putting on different components of the orthopedic brace or may be attached thereafter where the upper secondary straps at the upper portion of the intermediary brace are connected to the lumbar support back brace and the lower secondary straps of the intermediary brace are connected to the calf cuff. These straps may be tightened as shown to provide appropriate tension to the leg to alleviate sciatica (operation 1140).

In the foregoing description, the invention is described with reference to specific exemplary embodiments thereof. However, it will be evident that various modifications and changes may be made thereto without departing from the broader spirit and scope of the invention as set forth in the appended claims.

What is claimed is:

1. An orthopedic brace, comprising:
a base material configured to be formed for placement over a knee of a patient;
a first de-tensioning assembly positioned to be situated on a first side of the base material when positioned around the knee of the patient; and
a second de-tensioning assembly positioned to be situated on a second side of the base material when positioned around the knee of the patient,
wherein the first de-tensioning assembly includes a first elastic strap extending along the first side and the second de-tensioning assembly includes a second elastic strap extending along the second side.

2. The orthopedic brace of claim 1, wherein the first side is a medial side and a second side is a lateral side.

3. The orthopedic brace of claim 1, wherein the first de-tensioning assembly comprises a first strap coupled to a first end of the first elastic strap and terminated by a first connector and a second strap coupled to a second end of the first elastic strap terminated by a second connector.

4. The orthopedic brace of claim 3, wherein first connector is coupled to a fastening member extending from an anterior surface of a lumbar support back brace.

5. The orthopedic brace of claim 4, wherein the second connector is coupled to a fastening member positioned on a posterior side of a lower leg brace that is separate and distinct from the base material operating as a knee brace.

6. The orthopedic brace of claim 5, wherein the lower leg brace is a calf cuff.

7. The orthopedic brace of claim 1, wherein the first de-tensioning assembly comprises a strap for coupling to a calf cuff integrated as part of the orthopedic brace.

8. The orthopedic brace of claim 5, wherein the lower leg brace is an ankle support orthosis.

9. The orthopedic brace of claim 1, wherein, when worn, the first elastic strap is positioned along a medial side of the knee and the second elastic strap is positioned on a lateral side of the knee to provide balanced support on both sides of a leg of the patient.

10. The orthopedic brace of claim 1, wherein both the first de-tensioning assembly and the second de-tensioning assembly, when the orthopedic brace is worn, are located anterior to a hip joint anatomical axis of the patient and posterior to an anatomical knee joint axis.

11. The orthopedic brace of claim 3, wherein the second de-tensioning assembly comprises a third strap coupled to a first end of the second elastic strap and terminated by a third connector and a fourth strap coupled to a second end of the second elastic strap terminated by a fourth connector.

12. The orthopedic brace of claim 11, wherein the second connector is coupled to a first fastening member positioned on a posterior side of a lower leg brace and the fourth connector is coupled to a second fastening member positioned on the posterior side of the lower leg brace.

13. The orthopedic brace of claim 12, wherein (i) the second connector, coupled to the second strap, is positioned along a medial side and is coupled to the first fastening member positioned proximate to a lateral side, and (ii) the fourth connector, coupled to the fourth strap, is positioned along the lateral side and is coupled to the second fastening member positioned proximate to the medial side to form a crisscross pattern.

14. An orthopedic brace, comprising:
a knee brace that comprises a base material configured to be formed for placement over a knee of a patient, a first de-tensioning assembly situated on a first side of the base material and including a first elastic strap extending along the first side, a second de-tensioning assembly positioned to be situated on a second side of the base material, and including a second elastic strap extending along the second side; and
a lower leg brace coupled to the knee brace via the first elastic strap and the second elastic strap.

15. The orthopedic brace of claim 14, wherein the first side of the knee brace is a medial side and a second side of the knee brace is a lateral side.

16. The orthopedic brace of claim 14, wherein the first de-tensioning assembly comprises a first strap coupled to a first end of the first elastic strap and terminated by a first connector and a second strap coupled to a second end of the first elastic strap terminated by a second connector.

17. The orthopedic brace of claim 16 further comprises a lumbar support back brace, wherein the first connector is coupled to a fastening member extending from an anterior surface of the lumbar support back brace.

18. The orthopedic brace of claim 17, wherein the second connector is coupled to a fastening member positioned on a posterior side of the lower leg brace, the lower leg brace being a calf cuff or an ankle support orthosis.

19. The orthopedic brace of claim 14, wherein the first elastic strap is situated, when worn, along a medial side of the knee of the patient and the second elastic strap is situated along a lateral side of the knee to provide balanced support on both sides of a leg of the patient.

20. An orthopedic brace, comprising:
a knee brace that comprises (i) a base material configured to be formed for placement over a knee of a patient, (ii) a first de-tensioning assembly situated on a first side of the base material and including a first elastic strap extending along the first side, and (iii) a second de-tensioning assembly positioned to be situated on a second side of the base material and including a second elastic strap extending along the second side;
a lumbar support back brace including a first fastening member and a second fastening member extending from an anterior surface of the lumbar support back brace, the first fastening member for coupling to a first end of the first elastic strap and the second fastening member for coupling to a first end of the second elastic strap; and
a lower leg brace including a third fastening member for coupling to a second end of the first elastic strap and a fourth fastening member for coupling to a second end of the second elastic strap.

21. The orthopedic brace of claim 20, wherein the lower leg brace corresponds to either a calf cuff or an ankle support orthosis.
